(19) United States
(12) Patent Application Publication
(10) Pub. No.: US 2004/0067593 A1
(43) Pub. Date: Apr. 8, 2004

(54) Electron paramagnetic resonance method for diagnosis of active nephritis

(76) Inventors: Paul G Winyard, London (GB); Rong Guo, London (GB); Martin Raftery, London (GB); David D'Cruz, London (GB); Magdi Yaqoob, London (GB)

Correspondence Address: DILWORTH PAXSON LLP, 3200 MELLON BANK CENTER, 1735 MARKET STREET, PHILADELPHIA, PA, 19103, US

(21) Appl. No.: 10/398,989

(22) PCT Filed: Oct. 10, 2001
(86) PCT No.: PCT/GB01/04522

(30) Foreign Application Priority Data
Oct. 11, 2000 (GB) 00249383

(51) Int. Cl. G01N 33/48

(52) U.S. Cl. 436/63

(57) ABSTRACT

The invention relates to a method diagnosing active nephritis in a patient by using electron paramagnetic resonance spectroscopy to measure the amount of a spin trapping agent which can be converted to free radicals by a urine sample taken from the patient.

The present invention relates to a method of diagnosing active nephritis in a patient.

The kidney is concerned with excretion of waste products, maintenance of the constancy of the body's internal environment and in the biosynthesis of hormones. The importance of these functions is readily appreciated in kidney disease and the difficulties in providing effective artificial dialysis treatment (J. Cunningham in Textbook of Medicine, Second edition, pages 785-841, Eds. Souhami & Moxham, Churchill Livingstone (1994).

Nephritis is a broad diagnosis for a number of inflammatory conditions that affect glomerular and tubular regions of the kidney. This may present as proteinuria, haematuria, nephritic sediment where granular casts and fragmented red cells are present and abnormal glomerular filtration rate (GFR) which is greatly reduced in renal failure. Nephritis or “kidney inflammation” may ultimately lead to renal failure which is an outcome of many serious diseases. Renal failure is associated with structural abnormalities and functional tissue loss in the kidney which leads to uraemia. Briefly, uraemia can be characterised as the accumulation of toxic waste products in the body due to renal failure. Depletion of essential compounds and failure of biosynthetic functions of the kidney also contribute to this condition.

Nephritis in a patient may be described as being either active or inactive. The condition of nephritis can vary over time with greater or lesser degrees of severity and clinical symptoms. A patient is described as having inactive nephritis if they do not show clinical signs of an overt inflammatory response at a particular time, even though they are known to have had episode(s) of nephritis in the past. For example at a first consultation with a doctor, a patient may have haematuria (blood in the urine) which is associated with active nephritis, but at a second subsequent consultation they do not have haematuria, i.e. they have inactive nephritis.

Currently, in clinical practice, active nephritis is detected by the presence of haemoglobin or protein in the urine, and more reliably, nephritic sediment where granular casts and fragmented red cells are present. However, all of these methods suffer from interference, such as menstrual periods, and the finding of nephritic sediment is observer dependent. These diagnostic indicators may not appear early enough to allow clinical intervention to try to prevent the nephritis. Presently, the only accurate and reliable way of detecting active nephritis is by renal biopsy. This is an invasive procedure associated with a risk of severe bleeding and cannot be performed frequently. There is therefore a need to provide a simple, effective diagnostic assay to test for active nephritis in a patient before the onset of chronic or acute renal failure. Such a test could enable earlier intervention with the possibility that expensive dialysis could be avoided and serious kidney damage prevented.

It has now been found that by measuring the total excretion of certain compounds in the 24 hour urine of patients with certain diseases, such as connective tissue diseases, the presence or absence of active nephritis can be diagnosed.

In addition, by monitoring total urinary excretion levels of these compounds in patients with active nephritis, the risk of developing renal failure and the response to treatment may be assessed.

The presence of these species in the urine of such patients can be a prognostic indicator of the likelihood of the patient subsequently developing renal failure.

According to a first aspect of the invention, there is provided a method of diagnosing the condition of active nephritis in a patient, the method comprising:

(a) obtaining a urine sample from a patient;

(b) admixing a spin trapping agent with the sample or a portion thereof; and

(c) using electron paramagnetic resonance (EPR) analysis to determine the amount of free radical product derived from the spin trapping agent in the sample;

wherein an increased amount of free radical product derived from the spin trapping agent in the urine sample compared with a control value is diagnostic for the condition of active nephritis in the patient.

Uraemic plasma and dialysate has been studied in patients with renal failure in a study of the oxidative causes of atherosclerosis (Roselaar et al Kidney International 48 199-206 (1995)). Such patients do not produce sufficient urine to permit any analysis of kidney function by urinary measurements. The presence of oxidants in the plasma of such patients was detected using EPR spectroscopy. Oxidising activity was detected by monitoring the one electron oxidation of a spin trap, 3,5-dibromo-4-nitrosobenzene sulphonate (DBNBS) to the putative radical cation, DBNBS^{●+}. Uraemia was seen to be associated with an accumulation of oxidants in the plasma of patients with renal failure compared to normal healthy individuals with implications for the development of atherosclerosis or other vascular disease in uraemic patients. However, in patients with active nephritis, none of the plasma samples show the presence of an oxidant species in contrast to the plasma of uraemic patients with renal failure.

There are various ways in which the method of the present invention can be carried out. For example, in a preferred embodiment, the urine sample obtained from the patient is a 24 hour urine sample of known volume. In this case, in step (b), the spin trapping agent will be mixed with a portion of the sample and step (c) of the method is carried out by:

(i) using electron paramagnetic resonance (EPR) analysis to determine the amount of free radical product derived from the spin trapping agent in the portion of the sample;

(ii) calculating the amount of free radical product which would be present in the whole 24 hour urine sample;

wherein an increased amount of free radical product derived from the spin trapping agent in the 24 hour urine sample from the patient compared to a control value is diagnostic for the condition of active nephritis in the patient.

Alternatively, however, the amount of free radical product in the sample may be compared with the amount of a marker substance which is excreted at a constant rate in the urine. In this case, the method may include the additional steps of:

(d) measuring the amount of marker substance in the sample; and

(e) calculating the ratio of free radical product to marker substance;

wherein an increase in the free radical product: marker substance ratio compared with a control value is diagnostic for the condition of active nephritis in the patient.

In the context of the present invention, the term “ratio of free radical product to marker substance” incorporates both

$$\frac{\text{free radical procuct}}{\text{marker substance}} \text{and} \frac{\text{marker substance}}{\text{free radical product}}$$

whereas “free radical product: marker substance ratio” signifies:

$$\frac{\text{free radical procuct}}{\text{marker substance}}$$

Clearly therefore, in the above method, a decrease in the marker substance: free radical product ratio would also be diagnostic for the condition of active nephritis in the patient.

This method may be used with a 24 hour urine sample of known volume but it is particularly useful when a random urine sample is used. This is because the urine volume varies depending upon the fluid intake of the patient. Therefore comparison of the amount of free radical product with the amount of a marker substance which is excreted at a constant rate leads to a more meaningful value than simply measuring the amount or the concentration of free radical product in the urine sample.

A suitable marker substance for use in this comparison is creatinine. As discussed in more detail below, the level of creatinine clearance in patients with active nephritis is within the normal range (see Example 1).

In the context of the present specification, the term “control value” refers either to a single value or to a range of values. The control value used will depend upon which particular embodiment of the method is employed and will be a measurement which is equivalent to the value measured in the method of the invention but obtained from at least one healthy volunteer. More usually, measurements from a large number of healthy volunteers, for example at least fifty, are used to obtain a control range which is the normal range for healthy subjects.

For example, in the case where the sample from the patient is a 24 hour urine sample of known volume, the control value may be a normal range obtained by measuring the total amount of free radical product in the 24 hour urine samples of healthy volunteers.

On the other hand, in the case where the ratio of free radical product to marker substance is calculated, the control value may be a ratio range calculated from measurements in a number of healthy volunteers.

In the context of the present specification, a 24 hour urine sample is the total amount of urine excreted by a patient over a 24 hour period.

In the context of the present specification, a spin trapping agent is an agent which is capable of reacting with an agent present in the urine of patients with active nephritis to produce a charged or uncharged free radical product which is paramagnetic and sufficiently stable to be detected by EPR spectroscopy. The unpaired electron of the radical product can be detected by EPR spectroscopy, which is also known as electron spin resonance (ESR) spectroscopy.

The free radical product may be a direct reaction product of the spin trapping agent and the agent present in the urine sample. However, this is not necessarily the case and with some spin trapping agents, the free radical product will be derived indirectly.

Suitable spin trapping agents include DBNBS, which is thought to be converted to the radical cation DBNBS^{●+}. Analogues of DBNBS may also be used and these include isotopically labelled forms such as deuterium labelled DBNBS (DBNBS-d₂), ¹⁵N labelled DBNBS (DBNBS-¹⁵N) and deuterium and ¹⁵N double labelled DBNBS (DBNBS-d₂-¹⁵N). Other derivatives of DBNBS may also be used, for example 3,5-dichloro-4-nitrosobenzene sulphonate (DCNBS), which is disclosed in UK Patent Application No. 0030278.6 filed on Dec. 13, 2000 in the name of Randox Laboratories Limited. DCNBS provides similar sensitivity to DBNBS but has a better solubility.

Alternative spin trapping agents which are of use in the method of this invention include 5-(diethoxyphosphoryl)-5-methyl-1-pyrroline N-oxide (DEPMPO), 5,5-dimethyl-1-pyrroline-N-oxide (DMPO), N-tert-butyl-α-phenylnitrone (PBN), nitromethane or an iron (II) complex of N-methyl-D-glucamine dithiocarbamate (MGD) or diethyldithiocarbamate (DETC) or a derivative or analogue (including a labelled analogue) of one of these. Other spin trapping agents may also be used and suitable agents could easily be identified by those skilled in the art.

The most likely explanation for the conversion of the spin trapping agent to a free radical in the method of the present invention is that the urine sample contains one or more oxidising species. If this is, indeed, the case then the measurement of the conversion of the spin trapping agent to a free radical product is, in effect, a measurement of oxidising activity present in the 24 hour urine sample. In view of this, the concentration of free radical product is, in some cases, described below as the level of oxidising activity in the sample.

However, it should be stressed that even if the conversion of the spin trapping agent to free radical product does not occur because of the presence of an oxidising agent in the sample, this in no way prevents the method of the present invention from working effectively.

Methods in accordance with the present invention may be particularly useful in diagnosing active nephritis in patients who have renal impairment and have not yet developed renal failure, especially in patients suffering from connective tissue diseases, such as for example, Systemic Lupus Erythematosus (SLE), Churg-Strauss Syndrome (CSS) and Wegener's Granulomatosis (WG).

The method of the present invention is of great assistance in the diagnosis of other diseases associated with renal impairment, especially in distinguishing inflammatory renal disease from non-inflammatory renal disease. For example, the method could be used in SLE patients who develop renal impairment during pregnancy. In this situation, the method would be used to distinguish the underlying cause of the renal impairment, which could either be lupus nephritis or pre-eclamptic toxaemia. In addition, it may be possible to distinguish other non-inflammatory causes of renal impairment such as diabetes, hypertension or renovascular disease from inflammatory causes of renal impairment such as pyelonephritis in association with urinary tract infections.

A rapid increase in the amount of spin trapping agent which can be converted into free radicals by the urine of such patients is indicative of impending renal failure and in patients receiving treatment for active nephritis, repeated measurements of urinary oxidant excretion could be useful in monitoring response to treatment.

Therefore, in a further aspect of the invention there is provided a method for predicting the risk of renal failure and/or assessing the response to treatment in a patient with active nephritis, the method comprising:

(a) obtaining a urine sample from a patient;

(b) admixing a spin trapping agent with the sample or a portion thereof; and

(c) using electron paramagnetic resonance (EPR) analysis to determine the amount of free radical product derived from the spin trapping agent in the sample;

(d) repeating steps (a) to (c) at intervals;

wherein an increased amount of free radical product in the urine sample from the patient compared to one or more previous samples from the same individual is predictive for the condition of impending renal failure in the patient.

According to this aspect of the invention, repeated measurements of the oxidant excretion would be carried out. The interval between measurements may be from two days to two months depending upon the condition of the patient and the level of oxidant present at the first measurement.

As with the method of the first aspect of the invention, it is greatly preferred that the sample obtained from the patient is a 24 hour urine sample of known volume and the spin trapping agent is mixed with a portion of this sample. In this case, step (c) of the method is carried out by:

(i) using electron paramagnetic resonance (EPR) analysis to determine the amount of free radical product derived from the spin trapping agent in the portion of the sample; and

(ii) calculating the amount of free radical product which would be present in the whole 24 hour urine sample.

Alternatively, again as with the first aspect of the invention, it is also possible to compare the amount of free radical product in the sample with the amount of a marker substance which is excreted at a constant rate in the urine. In this case, therefore, the method may include the additional steps of:

measuring the amount of marker substance in the sample; and

calculating the ratio of free radical product to marker substance.

In this case, an increase in the free radical product:marker substance ratio compared to one or more previous samples from the same individual is predictive for the condition of impending renal failure in the patient.

As in the case of the first aspect of the invention, the measurement of the ration of free radical product to marker substance may be used with a 24 hour urine sample of known volume but is particularly useful when a random urine sample is used.

As mentioned above, creatinine is a suitable marker substance for use in this method.

The urine samples for analysis may be collected in the normal manner from the patient. The level of spin trapping agent converted to free radical may be assayed according to the method described in Roselaar et al (Kidney International 48 199-206 (1995)). For example, measured amounts of DBNBS and phosphate buffered saline (e.g. pH 7.4) may be admixed with the sample in a sample collector. After admixing the urine sample in the sample collector with the other components, the sample can then be transferred to an EPR analyser, for example a JEOL RE1X spectrometer.

It may also be convenient to assay for the level of renal function in the patient. Methods in accordance with this aspect of the invention can therefore also include assays of creatinine clearance. Plasma creatinine is an established marker of renal function. Both plasma creatinine and urinary creatinine can be measured by spectrophotometric methods, e.g. using the Sigma Diagnostic Kit “Creatinine 555” (Sigma-Aldrich, Poole, United Kingdom). Blood plasma can be collected from the patient in the normal manner. Creatinine clearance as an index of the kidney's ability to “clear” creatinine from blood can be calculated by the equation:

$$\text{Creatinine clearance} = \frac{\text{urine creatinine concentration} \times \text{urine flow rate}}{\text{plasma creatinine concentration}}$$

Indicators of inflammation which may be assessed in conjunction with the method of the present invention include the level of serum C-reactive protein and blood erythrocyte sedimentation rate.

According to another aspect of the invention there is provided a kit for the diagnosis of the condition of active nephritis in a patient, the kit comprising a spin trapping agent such as DBNBS or one of its derivatives or analogues mentioned above.

For example, the kit may comprise measured amounts of DBNBS, phosphate buffered saline (e.g. at pH 7.4) and a sample collector. After admixing the urine sample in the sample collector with the other components, the sample can then be transferred to an EPR analyser, e.g. a JEOL RE1X spectrometer. The kit may further include standards and/or a control sample.

According to a further aspect of the invention there is provided the use of a spin trapping agent such as DBNBS or the other spin trapping agents mentioned above in the diagnosis of the condition of active nephritis in a patient.

Preferred features for the second and subsequent aspects of the invention are as for the first aspect mutatis mutandis.

The invention will now be further described by way of reference to the following Examples and figures which are provided for the purposes of illustration only and are not to be construed as being limiting on the invention. Reference is made to a number of figures in which:

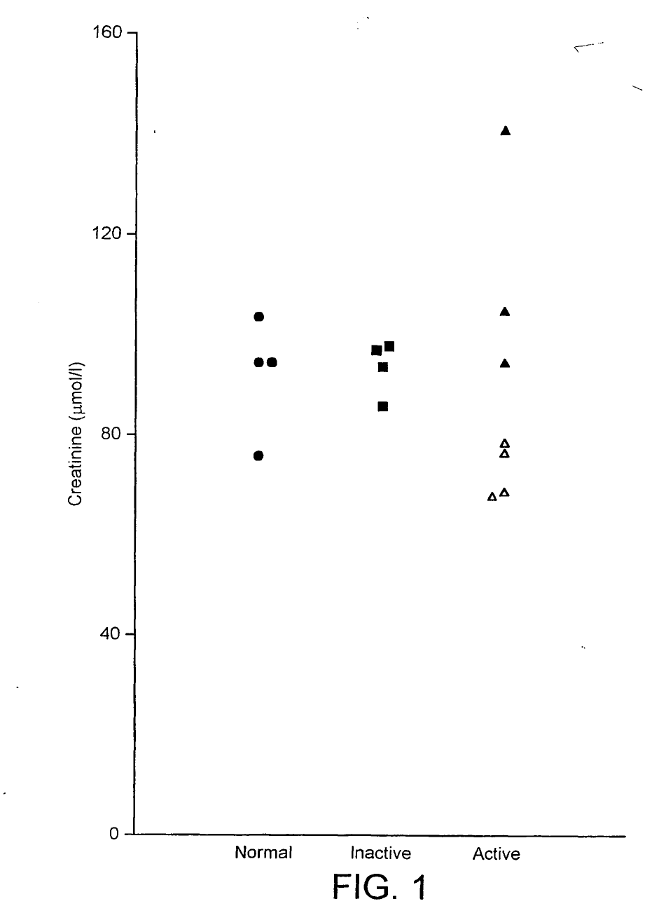

FIG. 1 shows creatinine concentration in serum of normal individuals and patients with inactive and active nephritis. The hollow symbols are from the same patient at different time points.

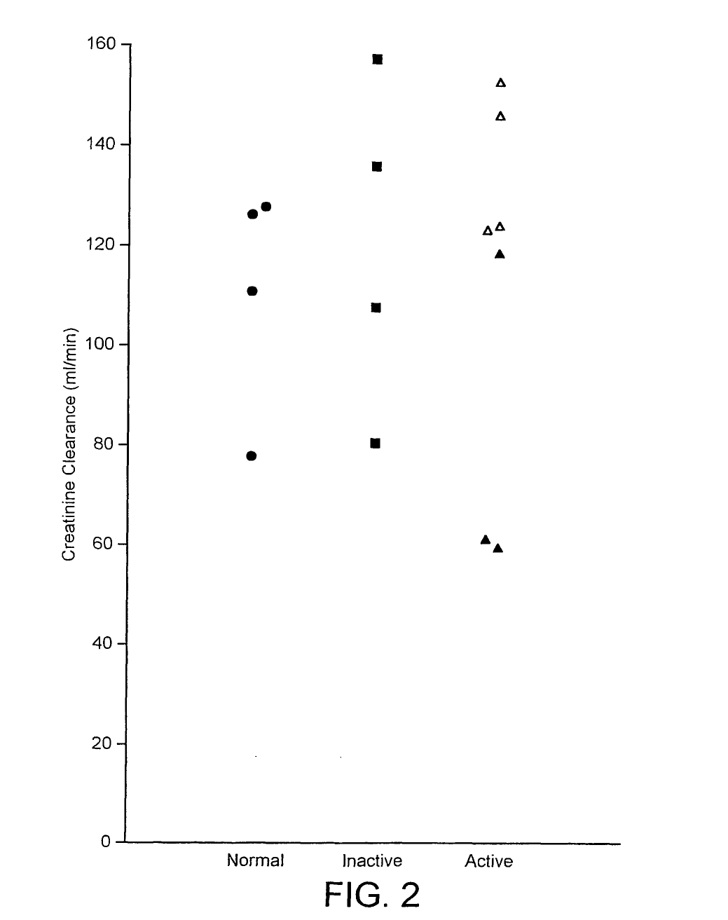

FIG. 2 shows creatinine clearance of normal individuals and patients with inactive and active nephritis. The hollow symbols are from the same patient at different time points.

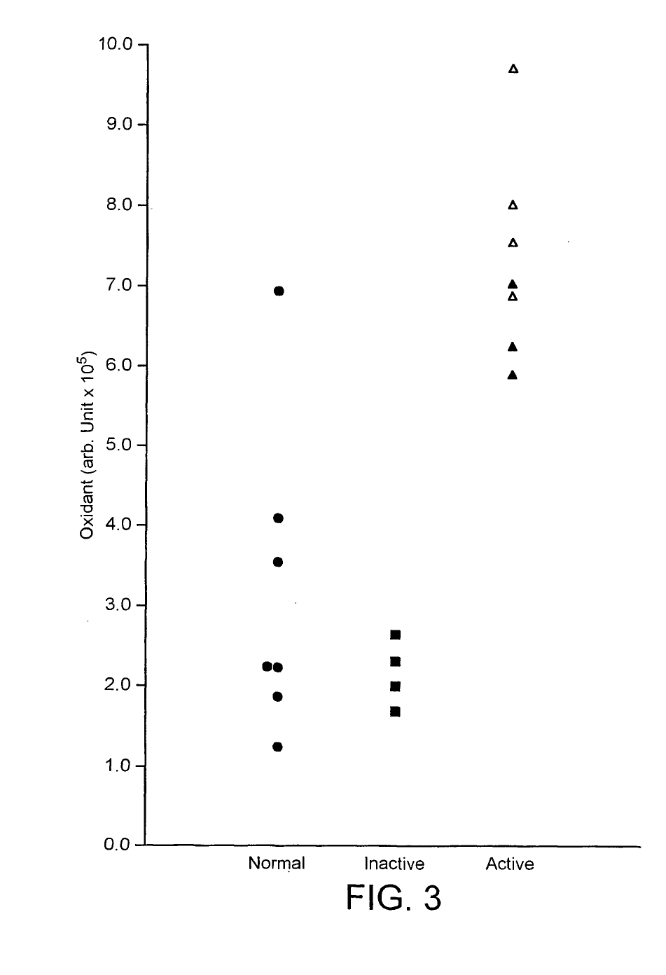

FIG. 3 shows total urinary oxidant excretion over 24 hours in normal individuals and patients with inactive and active nephritis.

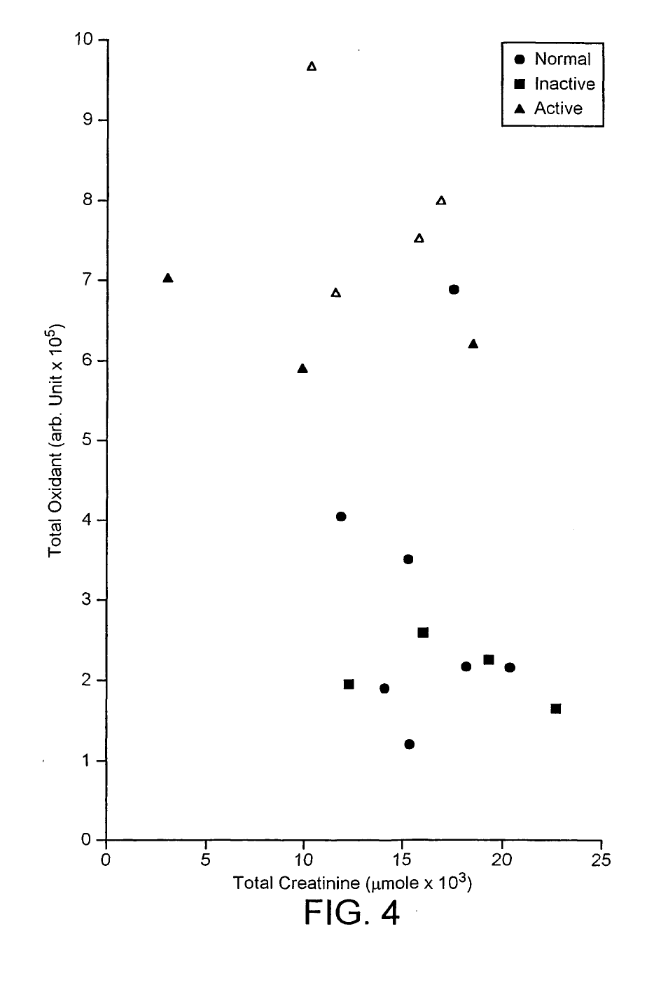

FIG. 4 shows the lack of association between total oxidant and creatinine in 24 hour urine of normal individuals and patients with inactive and active nephritis. The hollow symbols are from the same patient at a different time point.

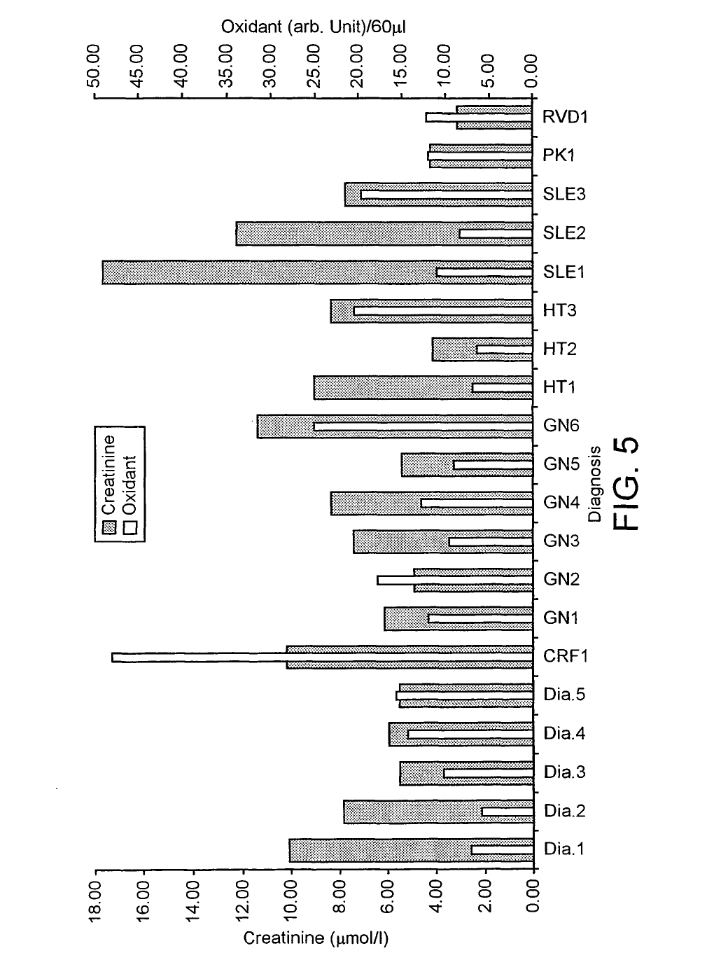

FIG. 5 is a plot showing oxidant and creatinine concentrations in random urine samples from patients with various diseases. Dia=diabetes; HT=hypertension; CRF=chronic renal failure; GN=glomerulonephritis; PK=polycystic kidneys; SLE=Systemic Lupus Erythematosus; RVD=renovascular disease.

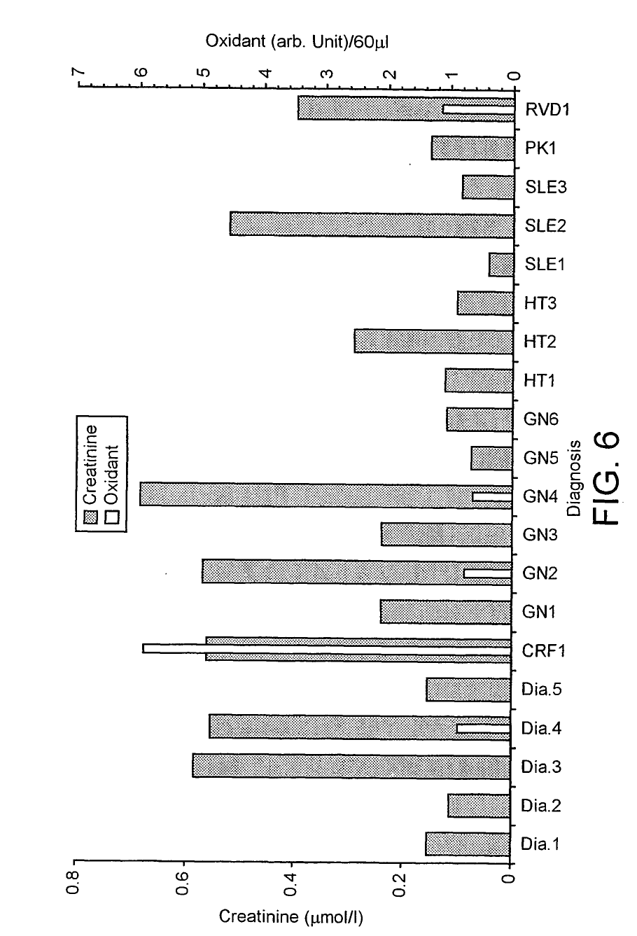

FIG. 6 is a plot showing plasma oxidant and creatinine levels in patients with various diseases. The abbreviations used are the same as for FIG. 5.

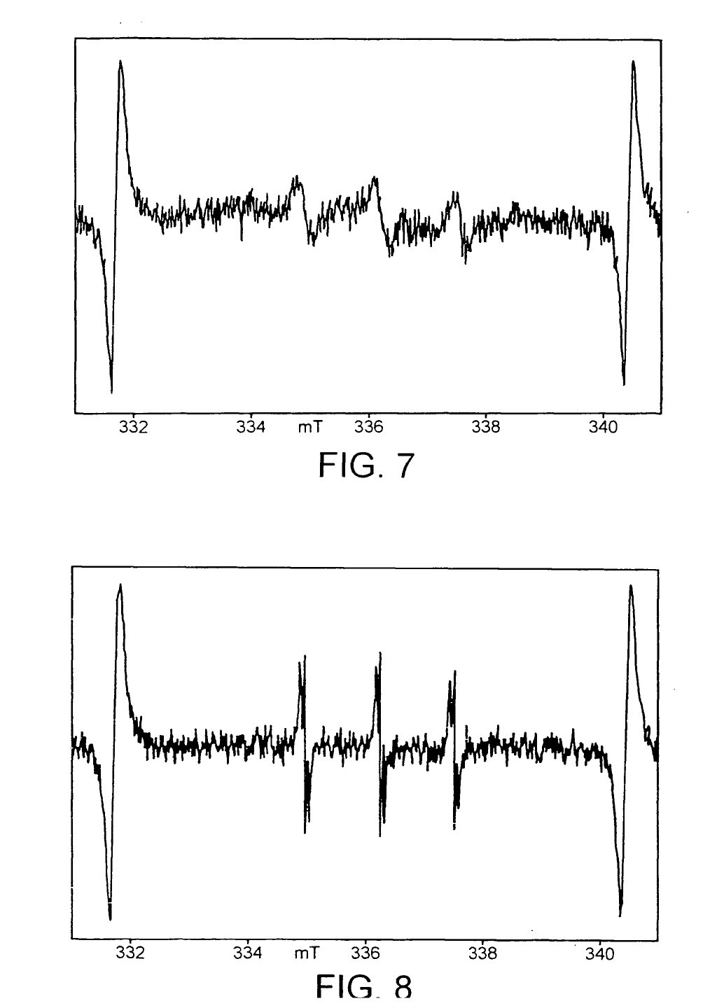

FIG. 7 shows the EPR spectrum of the DBNBS radical cation obtained from the reaction mixture of DBNBS with plasma of a patient with renal failure.

FIG. 8 shows the EPR spectrum of the DBNBS-SO₃^{●-} product obtained from the reaction mixture of DBNBS with hydrogen peroxide and horseradish peroxidase.

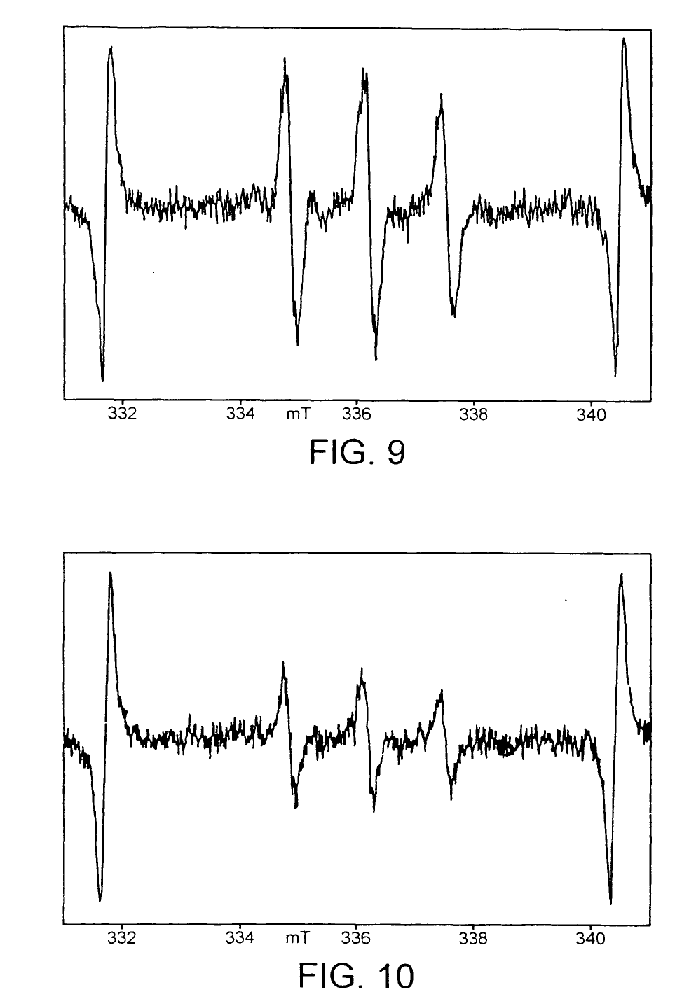

FIG. 9 shows the EPR spectrum of the DBNBS radical cation obtained from the reaction mixture of DBNBS with urine of a normal individual.

FIG. 10 shows the EPR spectrum of the same mixture as indicated in FIG. 9, with the addition of 10 mM dipotassium sulphite. The total reaction volume had been corrected by PBS to allow direct comparison of signal height with FIG. 9.

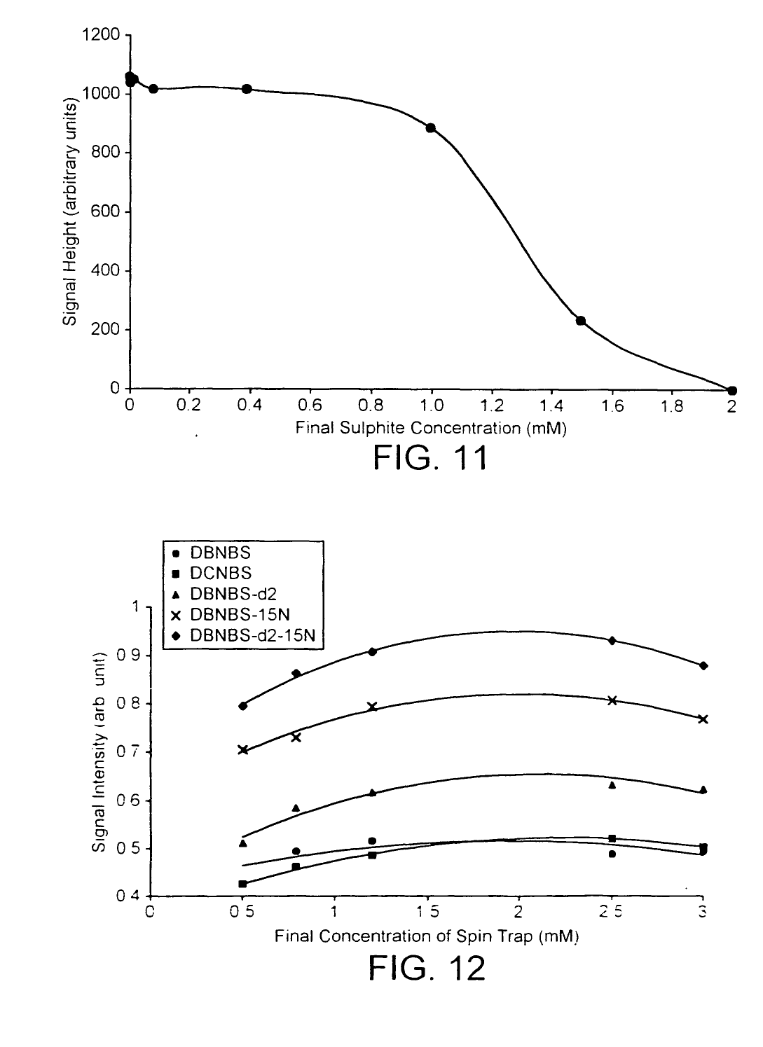

FIG. 11 shows the effect of sulphite concentration on the EPR signal obtained when DBNBS is reacted with normal human urine.

FIG. 12 shows the sensitivity of DBNBS analogues in the oxidant system.

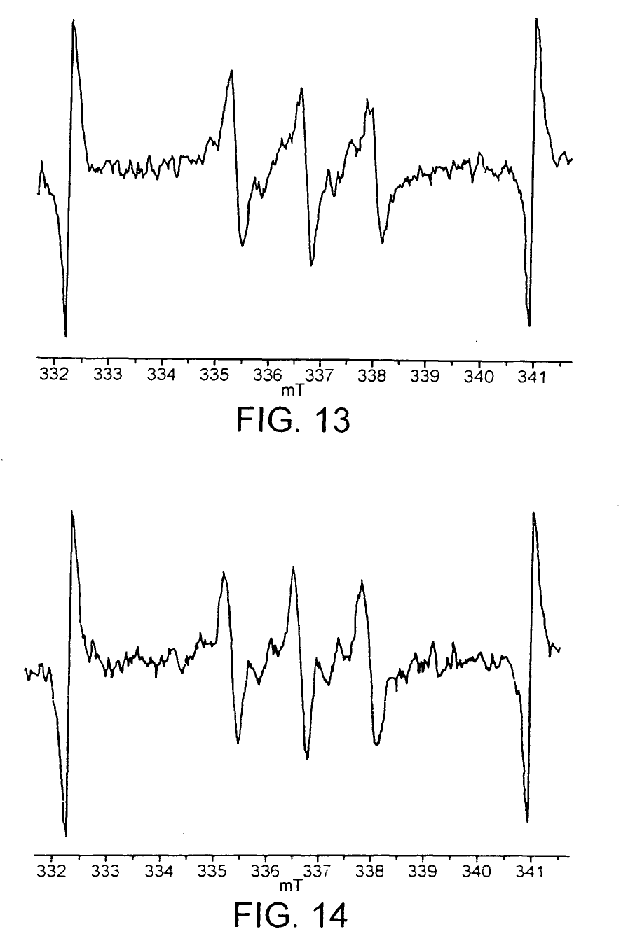

FIG. 13 is an EPR spectrum of DBNBS (1.2 mM) reacting with dialysate.

FIG. 14 is an EPR spectrum of DCNBS (2.5 mM) reacting with dialysate.

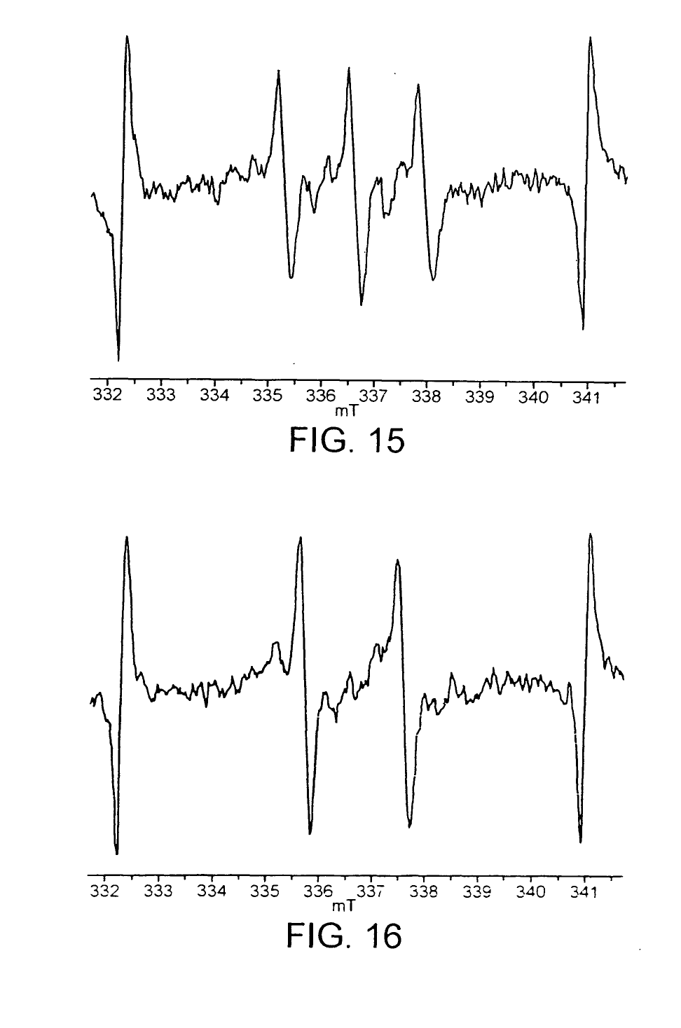

FIG. 15 is an EPR spectrum of DBNBS-d₂ (2.5 mM) reacting with dialysate.

FIG. 16 is an EPR spectrum of DBNBS-¹⁵N (2.5 mM) reacting with dialysate.

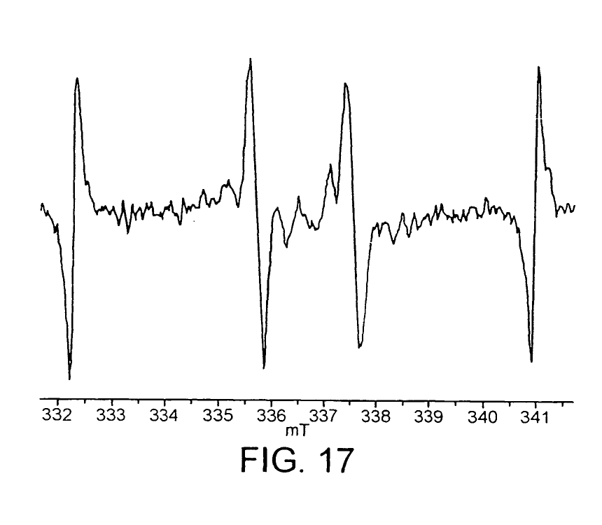

FIG. 17 is an EPR spectrum of DBNBS-d₂-¹⁵N (2.51 mM) reacting with dialysate.

### EXAMPLE 1

### Relationship between Conversion of Spin Trapping Agent to Radical and Creatinine Level in Urine and Plasma from Patients with Connective Tissue Diseases who have Active or Inactive Nephritis

The total amount of agent capable of converting a spin trapping agent to free radicals (here described as oxidising activity) in the urine of patients with connective tissue diseases who had not developed renal failure but had been clinically diagnosed with active or inactive nephritis was studied.

Serum and 24 hour urine was collected by Dr David D'Cruz (Royal London Hospital, Whitechapel) from five patients with SLE (samples were collected from one of the patients at four different time points), two with Churg-Strauss Syndrome (CSS, one of them had no nephritis) and one with Wegener's Granulomatosis (WG). All patients had normal renal function. Seven healthy volunteers were also included as controls. Four of these healthy volunteers had both serum and 24 hour urine collected and three of them had only 24 hour urine collected.

The amount of oxidising activity was determined by mixing 60 μl of urine or serum with 20 μl of phosphate buffered saline, pH 7.4, and 20 μl of 10 mM DBNBS. After a 25 minute incubation at room temperature, the oxidising activity was determined by EPR spectrometry (Jeol RE1X spectrometer), as described previously (Roselaar et al., 1995). Plasma creatinine (as an established marker of renal function) and urinary creatinine were measured by a manual spectrophotometric method (Sigma Diagnostic Kit “Creatinine 555”). Creatinine clearance (as an index of the kidney's ability to “clear” creatinine from blood) was calculated by the equation:

$$\text{Creatinine clearance} = \frac{\text{urinary creatinine concentration} \times \text{urine flow rate}}{\text{plasma creatinine concentration}}$$

The results showed that the serum creatinine concentrations in all patients were within the normal range (FIG. 1) and the creatinine clearances (FIG. 2) of all patients were within the normal range. However, the total oxidising activity in the 24 hour urine samples of the inactive versus active groups was different (FIG. 3). Statistical analysis (Students t-test) showed that the difference between the two groups was highly significant (p<0.0001; active nephritis: n=4, inactive nephritis: n=4). The difference in total oxidising activity between normal subjects and active nephritis patients was also significant (p<0.01). There was no significant difference between normal subjects and inactive nephritis patients (p>0.3). For the patient whose serum and 24 hour urine were collected at four different time points, the value of one particular sample, which was closest to the mean of the values obtained from the four samples was taken into account in the statistical calculation.

The lack of any obvious correlation between total urinary creatinine excretion and total urinary oxidising activity (FIG. 4) showed that regardless of the creatinine level the oxidising activity was higher in all patients with active nephritis and lower in all patients with inactive nephritis. This indicated that the total urinary oxidising activity could be an indicator of active nephritis in connective tissue diseases.

### EXAMPLE 2

### Oxidant in Urine and Plasma from Patients with Various Diseases in which there was Renal Involvement

The oxidising activity in the plasma and urine from patients with different diseases and without renal failure, or with different degrees of renal failure who had not been treated by dialysis, was investigated as follows.

Samples from twenty patients were collected from renal clinics at the Royal London Hospital, Whitechapel, in collaboration with Drs Magdi Yaqoob and Martin Raftery. Random urine and plasma were taken at the same time. The patients included five diabetes patients (Dia; plasma creatinine 109-589 μmol/l), three with hypertension (HT; plasma creatinine 98-289 μmol/l), three with SLE (plasma creatinine 40-522 μmol/l), six glomerulonephritis patients (GN; plasma creatinine 71-682 μmol/l), one patient with chronic renal failure for an unknown reason (CRF; plasma creatinine 559 μmol/l), one polycystic kidney patient (PK; plasma creatinine 148 μmol/l), and one renovascular disease patient (RVD; plasma creatinine 394 μmol/l). The normal range of plasma creatinine is 70-123 μmol/l. The determination of the oxidising activity and creatinine was the same as described above.

The results are shown in FIGS. 5 and 6. There was no correlation (FIG. 5) between urinary oxidising activity (arbitary units/ml) and urinary creatinine concentration (correlation coefficient=0.16), again confirming the finding obtained in the above study from patients with connective tissue diseases. Only five of the twenty patients had detectable plasma oxidant (FIG. 6). All these five patients had a plasma creatinine concentration equal to or greater than 394 μmol/l (394, 552, 559, 573 and 682), consisting of two patients with glomerulonephritis, one patient with diabetes, one patient with renovascular disease and one with chronic renal function. The concentration of urinary oxidising activity was also higher in these patients. However, two other patients with diabetes and SLE with higher plasma creatinine levels (589 and 522 μmol/l, respectively) did not have detectable plasma oxidant. This suggests that the two assays might provide different clinical information, but studies with large patient numbers are needed.

### EXAMPLE 3

### Effect of DBNBS Sulphite Radical (DBNBS-SO₃^{●-}) on Determination of Oxidant Found in the Plasma of Patients with Renal Failure

The oxidant(s) which is present in the plasma of patients with renal failure can react with the spin trap DBNBS to form the DBNBS radical cation (Roselaar et al., 1995). This radical cation gives a typical 3 line EPR spectrum with a hyperfine splitting a_{N}=1.32 mT (FIG. 7). However, Ichimori and his co-workers (1993) suggested that the EPR signal detected in this case was DBNBS-SO₃^{●-}, not the DBNBS radical cation.

We have determined a method for distinguishing the DBNBS sulphite radical from the DBNBS radical cation and have assessed whether contamination with sulphite during the synthesis of DBNBS could be a problem for determination of the oxidising activity.

The characteristic spectrum of DBNBS-SO₃^{●-} was a 9 line signal with an a_{N}=1.32 mT and a_{H}=0.06 mT (FIG. 8). This was obtained by reacting DBNBS with hydrogen peroxide and horseradish peroxidase. The addition of 10 mM sulphite to the reaction mixture caused the signal to increase by over 100 fold in intensity. The signal could also be obtained by reacting DBNBS with sulphite alone. However, when the DBNBS was reacted with the plasma of patients with renal failure and the urine from a normal individual, only the 3 line signal with an a_{N}=1.32 mT was seen (FIGS. 7 & 9, respectively). The hydrogen hyperfine splitting seen in the spectrum of DBNBS-SO₃^{●-} was absent. The addition of 1 mM and 10 mM sulphite to the reaction mixture caused a decrease in signal height, instead of the expected increase if the signal had arisen from DBNBS-SO₃^{●-} (compare FIGS. 9 and 10). Therefore, we confirmed that DBNBS-SO₃^{●-} and the DBNBS radical cation generated by renal patients' plasma or normal urine were completely different radicals.

Addition of sulphite to the mixture of DBNBS and plasma or urine decreased the signal height of the DBNBS radical cation. This could affect the determination of the oxidant in biological samples if there was sulphite contamination in the DBNBS preparation or endogenous sulphite in the biological sample itself. Therefore the dose response effect on addition of sulphite to the reaction mixture was investigated. The results showed that a final concentration of 2 mM sulphite in the reaction mixture completely abolished the 3 line signal of the DBNBS radical cation (FIG. 11). However, a final concentration of 0.4 mM sulphite only caused a reduction of 6.1% in the signal height and 0.08 mM of sulphite caused a reduction of 1.4%. Therefore the effect of sulphite on the determination of the DBNBS radical cation is negligible at the concentrations of sulphite present in DBNBS preparations or in patients' samples, and this potential problem can be discounted.

### EXAMPLE 4

### Reaction of the Oxidant with DBNBS and Various Analogues

In this example, the spin traps used were DBNBS and its analogues, DCNBS, DBNBS-d₂, ¹⁵N-DBNBS and DBNBS-d₂-¹⁵N. The purpose of the experiment was to demonstrate that it is possible to detect the oxidant using a variety of different spin traps. Dialysate rather than urine was used in this experiment and therefore DBNBS was used as a control spin trap to demonstrate that dialysate contains the same oxidant found in urine.

10 mM solutions of the spin traps (5, 8, 12, 25 and 30 μl for final concentrations of 0.5, 0.8, 1.2, 2.5 and 3 mM in phosphate buffered saline (PBS)) were added to 60 μl of the dialysate. The difference in volume was comprised of PBS. The final volume was 100 μl. The reaction mixture was mixed thoroughly and analysed by EPR spectroscopy after 25 minutes incubation at room temperature. The EPR conditions used were: microwave power 4 mW, CF 336.7 mT, SW±5 mT, ST 150s, TC 0.3s, MW 0.2 mT. The results are listed in Table 1 and FIG. 12. The graph of FIG. 12 represents the mean of two experiments. The variation between the two experiments was less than 18% when comparing with the mean. FIGS. 13-17 are EPR spectra of DBNBS and the various analogues reacting with dialysate. In these figures, the two large outer lines are the manganese marker.

TABLE 1
Comparison of the sensitivity of DBNBS and its analogues
for detection of the oxidant in dialysate from a patient
with renal disease

| | Spin trap | | |
|---|---|---|---|
| | concentration | | % of DBNBS |
| Spin Trap | (mM) | Signal intensity | signal |
| DBNBS | 1.2 | 0.516 | 100.0 |
| DCNBS | 2.5 | 0.521 | 101.0 |
| DBNBS-d₂ | 2.5 | 0.629 | 122.0 |
| ¹⁵N-DBNBS | 2.5 | 0.808 | 156.5 |
| DBNBS-d₂-¹⁵N | 2.5 | 0.934 | 181.1 |

### REFERENCES

Roselaar S. E., Nazhat N. B., Winyard P. G. et al. (1995) Kidney International. 48:199-206.

Ichimori K., Arroyo C. M., Pronal et al., (1993) Free Rad. Res. Commun. 19:s129-139.

1. A method of diagnosing the condition of active nephritis in a patient, the method comprising:
(a) obtaining a urine sample from a patient;
(b) admixing a spin trapping agent with the sample or a portion thereof; and
(c) using electron paramagnetic resonance (EPR) analysis to determine the amount of free radical product derived from the spin trapping agent in the sample;
wherein an increased amount of free radical product derived from the spin trapping agent in the urine sample compared with a control value is diagnostic for the condition of active nephritis in the patient.

2. A method as claimed in claim 1, wherein:
the urine sample obtained from the patient is a 24 hour urine sample of known volume;
in step (b), the spin trapping agent is mixed with a portion of the sample; and
step (c) of the method is carried out by:
(i) using electron paramagnetic resonance (EPR) analysis to determine the amount of free radical product derived from the spin trapping agent in the portion of the sample; and
(ii) calculating the amount of free radical product which would be present in the whole 24 hour urine sample;
wherein an increased amount of free radical product derived from the spin trapping agent in the 24 hour urine sample from the patient compared to a control value is diagnostic for the condition of active nephritis in the patient.

3. A method as claimed in claim 1 or claim 2 comprising the additional steps of:
(d) measuring the amount of a marker substance in the sample; and
(e) calculating the ration of free radical product to marker substance;
wherein an increase in the free radical product: marker substance ratio compared with a control value is diagnostic for the condition of active nephritis in the patient.

4. A method of predicting the risk of renal failure and/or assessing the response to treatment in a patient with active nephritis, the method comprising:
(a) obtaining a urine sample from the patient;
(b) admixing a spin trapping agent with the sample or a portion thereof;
(c) using electron paramagnetic resonance (EPR) analysis to determine the amount of free radical product derived from the spin trapping agent in the sample; and
(d) repeating steps (a) to (c) at intervals;
wherein an increased amount of free radical product in the urine sample from the patient compared to one or more previous samples from the same individual is predictive for the condition of impending renal failure in the patient.

5. A method as claimed in claim 4, wherein:
the urine sample obtained from the patient is a 24 hour urine sample of known volume;
in step (b), the spin trapping agent is mixed with a portion of the sample; and step (c) of the method is carried out by:
(i) using electron paramagnetic resonance (EPR) analysis to determine the amount of free radical product derived from the spin trapping agent in the portion of the sample; and
(ii) calculating the amount of free radical product which would be present in the whole 24 hour urine sample.

6. A method as claimed in claim 4 or claim 5, comprising the additional steps of:
(c) (iii) measuring the amount of a marker substance in the sample; and
(iv) calculating the ratio of free radical product to marker substance;
where an increase in the free radical product: marker substance ratio compared to one or more previous samples from the same individual is predictive for the condition of impending renal failure in the patient.

7. A method as claimed in claim 3 or claim 6, wherein the marker substance is creatinine.

8. A method as claimed in any one of the preceding claims wherein the spin trapping agent is:
3,5-dibromo-4-nitrosobenzene sulphonate (DBNBS);
example 3,5-dichloro-4-nitrosobenzene sulphonate (DCNBS)
5-(diethoxyphosphoryl)-5-methyl-1-pyrroline N-oxide (DEPMPO);
5,5-dimethyl-1-pyrroline-N-oxide (DMPO);
N-tert-butyl-α-phenylnitrone (PBN);
nitromethane;
an iron (II) complex of N-methyl-D-glucamine dithiocarbamate (MGD)
an iron (H) complex of diethyldithiocarbamate (DETC); or
a derivative or an analogue of any of the above.

9. A method as claimed in claim 9, wherein the spin trapping agent is DBNBS, deuterium labelled DBNBS (DBNBS-d₂), ¹⁵N-labelled DBNBS (DBNBS-¹⁵N) or deuterium and ¹⁵N double labelled DBNBS (DBNBS-d₂-¹⁵N).

10. A method as claimed in any one of claims 1 to 9, further comprising measuring creatinine clearance to assess renal function.

11. A method as claimed in any one of claims 1 to 10, further comprising measuring an index of acute phase reaction, for example serum C-reactive protein concentration or blood erythrocyte sedimentation rate.
